(19) United States
(12) Patent Application Publication
(10) Pub. No.: US 2025/0092318 A1
(43) Pub. Date: Mar. 20, 2025

(54) SYSTEM AND METHOD FOR RECOVERING HYDROCARBON LIQUIDS FROM EOR GASEOUS PRODUCTION STREAMS

(71) Applicant: OCCIDENTAL OIL AND GAS CORPORATION, Houston, TX (US)

(72) Inventors: Hitesh SHAH, Richmond, TX (US); Andres S. LOPEZ, Katy, TX (US)

(21) Appl. No.: 18/728,915

(22) PCT Filed: Jan. 17, 2023
(86) PCT No.: PCT/US2023/060762

§ 371 Date: Jul. 15, 2024

Related U.S. Application Data
(60) Provisional application No. 63/299,568, filed on Jan. 14, 2022.

(51) Int. Cl. C10G 5/06; C10G 7/00

(52) U.S. Cl. CPC C10G 5/06; C10G 7/00; C10G 2400/28

(57) ABSTRACT

A process for recovering liquid hydrocarbons from a gaseous stream containing the liquid hydrocarbons, the process comprising (a) providing a dehydrated gaseous stream including carbon dioxide, methane, ethane, propane, and C4 and heavier hydrocarbons; (b) cooling the dehydrated gaseous stream to form a condensate stream including C4 and heavier hydrocarbons; (c) separating at least a portion of the carbon dioxide, methane and ethane from the condensate stream; and (d) distilling the condensate stream to thereby form a liquid hydrocarbon stream.

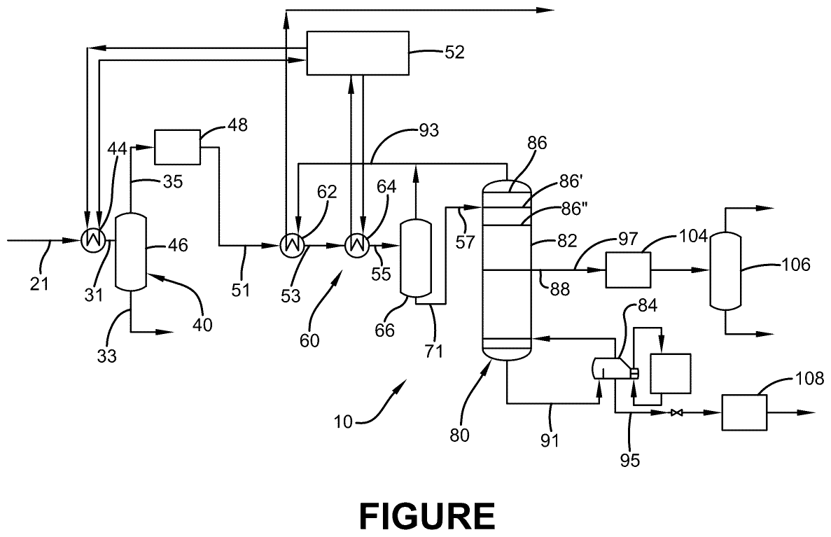

### FIELD OF THE INVENTION

Embodiments of the present invention provide a system and method for recovering hydrocarbon liquids from carbon dioxide-rich gaseous streams deriving from oil production streams such as production streams from enhanced oil recovery operations.

### BACKGROUND OF THE INVENTION

Gas streams that are associated with enhanced oil recovery (EOR) production streams general include hydrocarbons (e.g. methane and heavier hydrocarbon gases such as ethane generally up to octane), carbon dioxide, hydrogen sulfide, and nitrogen. Where carbon dioxide is used as the injected gas within an EOR operation, the associated gas stream (i.e. the gaseous component of the production stream) can also be relatively rich in carbon dioxide content. Despite having significant carbon dioxide content, the associated gas stream nonetheless includes valuable hydrocarbons. These hydrocarbons can be separated and sold as natural gas liquid (NGL), which generally includes C2 and heavier hydrocarbons, natural gasoline, which generally includes C4 and heavier hydrocarbons, and fuel gas, which generally includes C1 hydrocarbons. Other constituents, such as hydrogen sulfide (H2S) and nitrogen, are also commonly found in the associated gas streams.

Cryogenic extractive distillation is a common technique used to separate the hydrocarbons from carbon dioxide-rich associated gas streams. The systems employed for this process often include multi-column systems (e.g. up to 4-column systems), which are typically referred to as Ryan Holmes (RH) systems. Separation of carbon dioxide, C1 hydrocarbons, nitrogen, and a fraction of C2 and C3 hydrocarbons typically occurs within the first column. The light hydrocarbons are then separated as an overhead stream from the carbon dioxide within a second column. A third column, which is often referred to as a demethanizer, separates C1 hydrocarbons (along with nitrogen and a fraction of C2 and C3 hydrocarbons) as an overhead stream, and the bottoms of the column contains C4+ (due to the addition of additive at the tower overhead), which is combined with liquid produced from an additive recovery column (ARC). A fourth column, which may be referred to as a depropanizer or ARC, produces an overhead stream containing C2+ with carbon dioxide and hydrogen sulfide. The bottoms from the depropanizer or ARC produces lean oils (C4+) that can be used within the system to assist in separating azeotropic mixtures, particularly those that form between carbon dioxide and C2 hydrocarbons.

While these multi-column systems and processes provide several useful streams that have market value, these elaborate systems require rather large capital expenditures, and the conditions required to separate these various gases require rather large operating expenses.

In the face of these capital and operating expenses, simpler systems have been proposed. For example, single column systems have been proposed wherein the single column is adapted to separate C3, C4, and heavier hydrocarbons from the light-ends of the associated gas stream, which generally include C1-C2 hydrocarbons, carbon dioxide, and hydrogen sulfide. The overhead stream from this column can then be condensed using propane refrigeration to separate C3 hydrocarbons from the overhead stream. The recovered C3 hydrocarbons can be used to reflux the column. The remaining overheads, which include carbon dioxide, and C1 and C2 hydrocarbons, can then be reinjected into a reservoir.

### SUMMARY OF THE INVENTION

One or more embodiments of the present invention provide a process for recovering liquid hydrocarbons from a gaseous stream containing the liquid hydrocarbons, the process comprising (a) providing a dehydrated gaseous stream including carbon dioxide, methane, ethane, propane, and C4 and heavier hydrocarbons; (b) cooling the dehydrated gaseous stream to form a condensate stream including C4 and heavier hydrocarbons; (c) separating at least a portion of the carbon dioxide, methane and ethane from the condensate stream; and (d) distilling the condensate stream to thereby form a liquid hydrocarbon stream.

Yet other embodiments of the present invention provide a method for recovering liquid hydrocarbons from a gaseous stream containing the liquid hydrocarbons, the process comprising (a) providing a hydrated gaseous stream including water, carbon dioxide, methane, ethane, propane, and C4 and heavier hydrocarbons; (b) cooling the hydrated gaseous stream to condense a portion of the water within the hydrated gaseous stream to thereby form a cooled hydrated gaseous stream; (c) separating the condensed water from the cooled hydrated gaseous stream to provide a partially dehydrated stream; (d) dehydrating the partially dehydrated stream to provide a dehydrated gaseous stream including carbon dioxide, methane, ethane, propane, and C4 and higher hydrocarbon; (e) cooling the dehydrated gaseous stream to thereby form a condensate stream including C4 and heavier hydrocarbons; (f) separating a portion of the carbon dioxide, methane, ethane, and propane from the condensate stream; and (g) distilling the condensate stream to thereby form a liquid hydrocarbon stream.

### BRIEF DESCRIPTION OF THE DRAWINGS

The FIGURE is a schematic drawing of a system for recovering hydrocarbon liquids from a gaseous production stream according to embodiments of the invention.

### DETAILED DESCRIPTION OF ILLUSTRATIVE EMBODIMENTS

Embodiments of the invention are based, at least in part, on the discovery of a system and method for treating gaseous streams from oil production that includes cooling the stream by refrigeration and then subsequently distilling only a portion of the stream. In one or more embodiments, the cooled stream undergoes separation to remove at least a portion of the gaseous content of the stream, which thereby reduces the volume of the material that is subsequently distilled. While the prior art contemplates distillation techniques to recover one or more constituents of a gaseous oil production stream, practice of the present invention offers an advantage by separating appreciable volumes of gas prior to distillation. These advantages are realized in, among other things, reduced capital and operating expenses. In one or more sub-embodiments, refrigeration is also employed to cool the gaseous stream prior to dehydrating the stream. By cooling the stream prior to dehydration, an appreciable amount of water can be removed from the gaseous stream, which thereby alleviates capacity on the dehydration unit.

Generally speaking, one or more embodiments of the invention provide a system and method whereby carbon dioxide-rich gas streams flow through multiple cross-exchangers to thereby reduce the gas temperature and drop out liquids within a separator. The separated liquids flow to a distillation column (which may also be referred to as a column or a tower) where separation of liquids and gases takes place. The bottoms exiting the column include hydrocarbon liquids that are generally characterized by a reduced Reid Vapor Pressure (RVP). The overhead exiting the tower includes carbon dioxide, C1 hydrocarbon, C2 hydrocarbon, and a smaller fraction of C3 and heavier hydrocarbons. This overhead stream can be reinjected into a production field. Advantageously, the liquids produced at the bottom of the column can potentially be blended with oil at nearby central tank batteries (CTB) to recover economic value therefrom. Also, one or more streams can be recovered as side draws from the distillation column and sold as natural gas liquid (NGL), which generally includes C3 hydrocarbons, C4 hydrocarbons, and fractional amounts of heavier hydrocarbons. The overhead stream from the column, along with the overhead stream from the upstream separator, can be used to cool the inlet carbon dioxide-rich gas stream prior to further cooling by refrigeration.

### System Overview

Embodiments of the invention can be described with reference to the FIGURE, which shows oil recovery system 10 for treating a gaseous stream deriving from an oil production operation. This gaseous stream deriving from an oil production operation may be referred to as an inlet associated gas or simply as an inlet stream. System 10 generally includes a dehydration sub-system 40, separation sub-system 60, and distillation system 80. From an overview perspective, the inlet stream, which is carried by conduit 21, is treated within dehydration sub-system 40 to reduce the water content of the inlet stream to levels that can be tolerated by downstream cooling without the formation of hydrates (i.e. ice-like substances that form at elevated pressures and low temperatures because of an interaction between a liquid water phase and light natural gas components). Dehydration sub-system 40 produces a dehydrated stream that is carried by conduit 51 to separation sub-system 60. Within separation sub-system 60, the dehydrated stream is treated to separate appreciable amounts of gaseous constituents within the stream and thereby provide a liquid-rich stream that is carried by conduit 71 to distillation sub-system 80. The liquid-rich stream is treated within distillation subsystem 80 under conditions to produce a liquid bottoms stream having a desired volatiles content, which may be characterized by its Reid Vapor Pressure (ASTM D323). This liquid bottoms stream exits system 10 via conduit 95.

Volatile gases separated from separation sub-system 60 and distillation sub-system 80 may be routed out of system 10 via conduit 93.

In one or more embodiments, dehydration sub-system 40 includes a cooling unit 44 that is adapted to cool the gaseous inlet stream and produce a cooled inlet stream that is carried by conduit 31 to a separator 46. As shown, cooling unit 44 may obtain refrigeration requirements from a refrigeration unit 52, such as a propane refrigeration unit. As a result of cooling, at least a portion of the water within the inlet stream is condensed and therefore the cooled inlet stream carried by conduit 31 may include condensed water. The cooled inlet stream may also include condensed hydrocarbons since a fraction of the hydrocarbons within the inlet stream may also condense upon cooling. As the skilled person appreciates, the amount of hydrocarbons that may condense can depend on several factors including the nature of the hydrocarbons present as well as the richness of the gas stream.

Separator 46, which may also be referred to as knockout drum 46 or pre-cooling separator 46 or two-phase separator 46, is adapted to separate condensed water and remove the condensed water and any condensed hydrocarbons from system 10 via conduit 33. Separator 46 is also adapted to direct a partially-dehydrated gaseous stream to a dehydration unit 48 via conduit 35. The condensed water stream (as well as any condensed hydrocarbons therein), which is removed from separator 46 via conduit 33, can be routed to a central tank battery (CTB) for further treatment such as the removal of hydrocarbon liquids that may be contained therein. Dehydration unit 48 is adapted to further reduce the water content of the partially-dehydrated gaseous stream and thereby produce the dehydrated stream that is carried to separation sub-system 60 via conduit 51.

In one or more embodiments, separation sub-system 60 includes a cooling unit 64 that is adapted to cool the dehydrated stream and produce a partially condensed, dehydrated stream that is carried by conduit 55 to a separator 66. As shown, cooling unit 64 may obtain refrigeration requirements from a propane refrigeration unit 52. Separator 66, which may also be referred to as knockout drum 66 or inlet separator 66 or two-phase separator 66, is adapted to separate condensed constituents within the partially condensed, dehydrated stream and direct the condensed constituents to distillation sub-system 80 via conduit 71. Separator 46 is also adapted to direct the gaseous constituents of the partially condensed, dehydrated stream as an overhead stream and out of system 10 via conduit 93.

In one or more embodiments, separation sub-system 60 includes an optional pre-cooling unit 62 that is adapted to supply cooling to the dehydrated stream carried by conduit 51 prior to cooling within unit 64 and thereby produce a pre-cooled, dehydrated stream that is carried by conduit 53 to cooling unit 64. As shown, pre-cooling unit 62 may obtain cooling requirements (e.g. refrigeration) from a gaseous stream carried by conduit 93, which gases are derived from overhead streams from separator 66 and/or distillation system 80.

In one or more embodiments, distillation sub-system 80 includes a distillation tower 82 and a heating subsystem 84 adapted to heat liquid constituents within the bottom portion of tower 82. Conventional distillation towers may be used in practicing the present invention. As the skilled person will appreciate, tower 82 may include elements adapted to increase contact between gases and liquids within the tower; for example, tower 82 may include reflux trays 86, 86′, and 86″. The skilled person also appreciates that the contacting elements or media may include packing. Heating subsystem 84 may also be conventional in nature. As the skilled person appreciates, heating subsystem 84 may include a reboiler, which may include a shell and tube heat exchanger system. Reboiler 84 heats the bottoms from the column and returns the heated medium (e.g. vapor) to the tower via a circulation loop 91. According to embodiments of the invention, liquid hydrocarbon stream (which may also be referred to as a lean oil stream), which carried by conduit 95, can include condensate removed from reboiler 84.

In one or more embodiments, a cooling unit 108 may be positioned downstream of separation unit 80 (e.g. downstream of reboiler 84). Cooling supplied to the liquid hydrocarbon stream carried by conduit 95 can be supplied from conventional cooling techniques such as, but limited to, water cooling, air cooling, or refrigeration. As will be described further below, the liquid hydrocarbon stream (i.e. lean oil stream) can then be routed to a tank battery or pipeline.

Also, in one or more embodiments, tower 82 may include a side outlet 88, which may also be referred to as side draw 88, for removal of desired hydrocarbon streams (e.g. NGLs) from tower 82 via conduit 97. Gases exit tower 82 as an overhead gaseous stream and are routed out of system 10 via conduit 93.

In those embodiments where tower 82 includes a side outlet 88, hydrocarbons exiting tower 82 via side draw 88 can be further treated downstream. For example, the hydrocarbon side stream, which is carried by conduit 97, can be cooled. As those skilled in the art appreciate, the side stream may include a relatively high concentration of volatiles (e.g. C3 and C4 hydrocarbons that are above their vapor pressure within tower), and these hydrocarbons can be cooled to form a liquid hydrocarbon stream such as an NGL stream. For example, a cooling unit 104 can be positioned downstream of tower 82. Cooling can take place by using conventional techniques including water cooling, air cooling, or refrigeration. Additionally, the side stream (e.g. NGL stream) can undergo further downstream separation to further purify the stream. For example, a separator 106 can be positioned downstream of tower 82 and downstream of cooling unit 104. A conventional separator may be employed such as, but not limited to, as two-phase separator, which may also be referred to as a knockout separator.

The skilled person will appreciate that the various components of the systems described herein are in fluid communication via the various conduits that are described. This fluid communication may be direct or indirect, where indirect fluid communication incudes situations where other sub-units or sub-components may be positioned between components described as being in fluid communication. The skilled person will also appreciate that the various components will include inlets and outlets through which the various stream may enter and exit the components and enter the various conduits.

### Inlet Stream

As indicated herein, the inlet stream (which is carried to the system via conduit 21) is a gaseous stream deriving from an oil production stream such as an oil production stream obtained from a carbon dioxide flooded reservoir (i.e. carbon dioxide assisted enhanced oil recovery). The skilled person appreciates that the inlet stream may have undergone primary separation (e.g. at a satellite separation facility) that is adapted to separate liquids and gases. In one or more embodiments, this primary separation takes place at pressures of from about 75 to about 500 PSIG, in other embodiments from about 150 to about 450 PSIG, and in other embodiments from about 200 to about 400 PSIG.

In one or more embodiments, the inlet stream includes from about 30 to about 95 vol %, in other embodiments from about 60 to about 92 vol %, and in other embodiments from about 80 to about 91 vol % carbon dioxide.

In one or more embodiments, the inlet stream includes from about 0.30 to about 0.65 vol %, in other embodiments from about 0.40 to about 0.63 vol %, and in other embodiments from about 0.50 to about 0.61 vol % water. In one or more embodiments, the inlet stream is saturated with water, and the skilled person will therefore appreciate that the amount of water present will be a function of temperature and pressure of the incoming stream.

In one or more embodiments, the temperature of the inlet stream, which is prior to cooling at dehydration sub-system 40, is from about 30 to about 130° F., in other embodiment from about 40 to about 125° F., and in other embodiments from about 65 to about 120° F. (Fahrenheit).

In one or more embodiments, the pressure of the inlet stream, which is prior to cooling at dehydration sub-system 40, is from about 75 to about 500 PSIG, in other embodiment from about 150 to about 450 PSIG, and in other embodiments from about 200 to about 400 PSIG.

In one or more embodiments, the inlet stream includes carbon dioxide, methane, ethane, propane, and C4 or heavier hydrocarbons (e.g. butane, pentane, hexane, heptane, and octane). In one or more embodiments, the inlet stream includes hydrogen sulfide.

### Dehydration Sub-System

As described above, dehydration sub-system 40 is operated to reduce an appreciable amount of water from the inlet stream (i.e. the stream carried by conduit 21) prior to being treated within dehydration unit 48. In one or more embodiments, treatment within cooling unit 44 and separation within separator 46 reduces the water content of the inlet stream by greater than 40 vol %, in other embodiments by greater than 50 vol %, and in other embodiments by greater than 65 vol %, relative to the total water within the inlet stream. Inasmuch as the inlet stream is typically saturated (i.e. 100% of the water potential at a given temperature and pressure), the skilled person will appreciate that by reducing appreciable levels of water from the inlet stream, the requirements placed on dehydration unit 48 is greatly reduced, which in turn reduces capital and operating expenses.

In one or more embodiments, dehydration unit 48 is adapted to reduce the water levels within the partially-dehydrated stream down to levels of less than 100 ppm, in other embodiments less than 75 ppm, in other embodiments less than 45 ppm, in other embodiments less than 30 ppm, in other embodiments less than 20 ppm, and in other embodiments less than 1 ppm.

In one or more embodiments, dehydration sub-system 40 (i.e. cooling unit 44 and separator 46) cools the inlet stream to a temperature of from about 30 to about 110° F., in other embodiment from about 55 to about 100° F., in other embodiment from about 60 to about 110° F., and in other embodiments from about 65 to about 95° F. (Fahrenheit).

In one or more embodiments, dehydration unit 48 includes a liquid desiccant system. Conventional liquid desiccant systems can be employed when practicing the present invention. In one or more embodiments, the liquid desiccant is a glycol such as triethylene glycol, diethylene glycol, ethylene glycol, or tetraethylene glycol. As the skilled person appreciates, these systems typically include a desiccant regeneration unit that operates in conjunction with desiccant tower, which may also be referred to as a desiccant contactor.

### Separation Sub-System

As described above, separation sub-system 60 is operated to reduce an appreciable volume of material delivered to distillation system 80. In other words, an appreciable volume of the dehydrated gaseous stream entering separation sub-system 60 is removed from separator 66 as a gaseous stream thereby appreciably reducing the volume of material that is treated by distillation system 80. In one or more embodiments, greater than 85 vol %, in other embodiments greater than 90 vol %, in other embodiments greater than 98 vol %, in other embodiments by greater than 98.5 vol %, and in other embodiments greater than 99 vol % of the dehydrated gaseous stream entering separation system 60 is removed from separation unit 66 as a gaseous stream (i.e. it is removed from unit 66 as an overhead stream via conduit 93). Stated differently, less than 15 vol %, in other embodiments less than 10 vol %, in other embodiments less than 5 vol %, in other embodiments less than 3 vol %, in other embodiments less than 2 vol %, in other embodiments by less than 1.5 vol %, and in other embodiments less than 1 vol % of the dehydrated gaseous stream entering separation sub-system 60 is routed to distillation sub-system 80 (i.e. it is removed as a bottoms stream out of separation unit 66 via conduit 71), with the balance exiting the system as a gaseous stream from separator 66.

Inasmuch as the dehydrated stream that is delivered to separation sub-system 60 (via conduit 51) includes a large volume fraction of gas (e.g. typically includes greater than 98 vol % gaseous constituents), the skilled person will appreciate that by reducing appreciable levels of gas from the dehydrated stream, the requirements placed on distillation sub-system 80 (e.g. the size of distillation column 82) is greatly reduced, which in turn reduces capital and operating expenses.

In one or more embodiments, separation sub-system 60 (e.g. cooling unit 64 and optionally 62) cools the dehydrated stream, which is then delivered to separator 66, to a temperature of from about −40 to about 5° F., in other embodiment from about −30 to about 0° F., and in other embodiments from about −20 to about −5° F. (Fahrenheit).

### Distillation Sub-System

The skilled person readily appreciates the physical and operational parameters that are necessary to achieve desired results within a distillation column, such as distillation column 82. For example, the skilled person appreciates that the amount of heat supplied to the liquid bottoms and the pressure of the column impact the nature of the bottom liquid draw. In one or more embodiments, these parameters are chosen to achieve a desired level of volatiles within the liquids bottom draw of the distillation sub-system (i.e. sub-system 80). As indicated above, the desired level of volatiles can be measured by Reid Pressure Value (RVP), which is determined according to ASTM D-323. In one or more embodiments, distillation sub-system 80 is operated under conditions to provide an oil product (i.e. a bottom liquid draw exiting via conduit 91) characterized by an RVP (ASTM D-323) of from about 9 to about 30 PSIA, in other embodiments from about 15 to about 27 PSIA, and in other embodiments from about 19 to about 25 PSIA. In these or other embodiments, distillation sub-system 80 is operated under conditions to provide an oil product having an RVP (ASTM D-323) of less than 35 PSIA, in other embodiments less than 30 PSIA, in other embodiments less than 19 PSIA, and in other embodiments less than 12 PSIA. Advantageously, the present invention provides a mechanism whereby the skilled person, once having an appreciation of the present invention, can manipulate the inputs of the system and methods of the present invention to thereby produce both natural gas liquids (NGLs) and lean oil, or manipulate the inputs and produce only lean oil if desired. For example, the production of lean oil can be maximized by eliminating or limiting the amount of side draw taken from distillation tower 82.

### Hydrogen Sulfide Management

In one or more embodiments, hydrogen sulfide (H2S) within the inlet stream exits system 10 at separation sub-system 60 (i.e. separated with the gaseous stream from separator 66 via conduit 93). In these or other embodiments, hydrogen sulfide exits system 10 at distillation sub-system 80 (i.e. separated with the gaseous stream from distillation tower 82 via conduit 93). In other embodiments, distillation tower 82 is operated under conditions to allow for hydrogen sulfide to exit tower 82 at side draw 88. As the skilled person will appreciate, the amount of hydrogen sulfide separated via side draw 88 can depend on the relative recovery of C2/C3.

Integration with Central Tank Battery or Other Liquid Degasification

In one or more embodiments, the oil product recovered from distillation sub-system 80 (i.e. the bottom draw from column 82 routed via conduit 91) is blended with oil having a lower RVP (ASTM D-323). For example, the oil product can be blended with oil processed at a nearby oil processing facility such as central tank battery. The skilled person appreciates that an RVP value of less than 10 PSIA (ASTM D-323) is desirable, yet operating distillation sub-system 80 at conditions appropriate to achieve this RVP value might not be beneficial in all situations (e.g. require too much energy requirements), and therefore oil product having a higher RVP value (such as that provided by distillation sub-system 80) can beneficially be blended with oil having a lower RVP value (e.g. from a central tank battery). As the skilled person appreciates, produced oils from EOR operations are often characterized by an RVP of from about 3.0 to about 5.0, while industry targets are less than 10 or even 12 depending on climate. According to embodiments of this invention, lean oil obtained from the gas processing operations of the present invention, which can be operated to produce lean oil with higher RVP (e.g. an RVP of about 15 to about 27), can be proportionally blended with lower RVP oils (e.g. RVP of 3 to about 5) and thereby provide oil that is closer to industry acceptable RVP values.

Integration with Production Field

As noted above, the gaseous stream removed from the system of the present invention can be reinjected into a formation that is undergoing an enhanced oil recovery operation. As a result, hydrocarbons removed from the system (i.e. removed from system 10 via conduit 93) can be later recovered. The skilled person understands that the gaseous stream removed from system 10 may require compression prior to reinjection into an enhanced oil recovery operation.

For purposes of this specification, reference to gases, liquids, or solids refers to the state of a given material or composition at standard conditions of pressure and temperature. Also, the skilled person will appreciate that reference to hydrocarbons with the designation “C” followed by a number refers to a hydrocarbon including the designated number of carbon atoms. For example, C4 hydrocarbon, or simply C4, refers to the group of hydrocarbons including four carbon atoms including, but not limited to, n-butane and isobutene. The designation “+” refers to the designated hydrocarbon and higher hydrocarbons. For example, C4+ refers to those hydrocarbon that include four carbon atoms plus those hydrocarbons that include more than four carbon atoms.

### EXAMPLES

In order to demonstrate the practice of the present invention, the following examples have been prepared and tested. The examples should not, however, be viewed as limiting the scope of the invention. The claims will serve to define the invention.

In order to demonstrate practice of the present invention, examples were simulated using industry-accepted simulation software. As shown in Table I below, two cases are simulated. The first included about 50 mole % of carbon dioxide, and the second included about 85 mole % of carbon dioxide.

The system that was simulated was configured according to the system provided in the FIGURE, which as explained above includes a pre-cooling unit 44, a separator 46, a dehydration unit 48, a cooling unit 64, a separator 66, a distillation system 80 including distillation tower 82 and reboiler 84, and a separator 106. An inlet stream is carried by conduit 21 is cooled at pre-cooling unit 44 and introduced to separator 46, which produces a condensed stream carried by conduit 33 and a partially-dehydrated gaseous stream carried by conduit 35. The partially-dehydrated gaseous stream is then dehydrated within dehydration unit 48 to produce a partially-condensed, dehydrated stream that is cooled at cooling unit 64 and delivered to separator 66 via conduit 55. Cooling unit 64 is a shell and tube heat exchanger that receives cooling requirements from a propylene condenser 52. The liquid condensate from separator 66, which may be referred to as dehydrated condensate, is routed to distillation tower 82 via conduit 71, and the gaseous overhead from separator 66 is routed out of the system via conduit 93. Overhead from tower 82 is routed out of the system via conduit 93, a side-draw is routed out of tower 82 via conduit 97, and the bottoms, which may be referred to as lean oil, is routed out of tower system 80 via conduit 95.

### Example I

In the first example, where the inlet stream included about 50 mole % of carbon dioxide, tower 82 was configured with 22 ideal trays, the feed point was tray 1, the condenser duty (i.e. propylene refrigeration) was about 2 MMBtu/hr. (million British thermal units per hour), and the reboiler duty was about 0.9 MMBtu/hr. The bottom temperature of tower 82 was 273° F. The system was configured to receive the inlet stream at about 20 MMscfd (million standard cubic feet per day).

TABLE I

| | | | Condensed | Partially | | | | |
|---|---|---|---|---|---|---|---|---|
| | | Partially | Water | Condensed & | Dehydrated, | | Side Draw | Tower |
| Stream | Inlet | Dehydrated | Stream | Dehydrated | Condensed | Overhead | NGL | Bottoms |
| (Conduit #) | (21) | (35) | (33) | (55) | (71) | (93) | (97) | (95) |
| Rate | 20.07 | 20.03 | — | 20 | 0.85 | 19.6 | — | — |
| (MMscfd) | | | | | | | | |
| Temp (° F.) | 90 | 60 | 60 | −35 | −35 | −34 | 115 | 115 |
| Pressure | 214.7 | 204.7 | 204.7 | 185 | 185 | 182 | 173 | 174 |
| (PSIA) | | | | | | | | |
| Liquid (BPD) | — | — | 6.2 | — | 486 | — | 218 | 71 |
| RVP (PSIA) | — | — | — | — | — | — | 103 | 19 |

Chemical Composition (Mole %)

| | | | | | | | | |
|---|---|---|---|---|---|---|---|---|
| CO2 | 49.813 | 49.927 | 0.400 | 49.9988 | 31.0628 | 50.9874 | 0 | 0 |
| H2S | 1.488 | 1.492 | 0.040 | 1.4939 | 2.5291 | 1.5235 | 0.0001 | 0 |
| Nitrogen | 0.299 | 0.300 | 0 | 0.3000 | 0.0099 | 0.3059 | 0 | 0 |
| Methane | 35.326 | 35.407 | 0 | 35.4579 | 4.8610 | 36.1591 | 0.00 | 0 |
| Ethane | 6.847 | 6.863 | 0 | 6.8725 | 8.8089 | 7.0084 | 0.0002 | 0 |
| Propane | 3.870 | 3.879 | 0 | 3.8844 | 21.4755 | 3.2870 | 43.228 | 0.12 |
| i-Butane | 0.397 | 0.398 | 0 | 0.3984 | 4.7164 | 0.2163 | 11.860 | 1.24 |
| n-Butane | 1.092 | 1.094 | 0 | 1.0956 | 16.0176 | 0.4550 | 38.829 | 13.63 |
| i-Pentane | 0.099 | 0.099 | 0 | 0.0996 | 1.9710 | 0.0176 | 1.802 | 13.35 |
| n-Pentane | 0.298 | 0.298 | 0 | 0.2988 | 6.2629 | 0.0365 | 3.882 | 49.6 |
| n-Hexane | 0.099 | 0.099 | 0 | 0.0996 | 2.284 | 0.0030 | 0.398 | 22.1 |
| H2O | 0.370 | 0.144 | 99.56 | 0.0000 | 0 | 0 | 0.000 | 0 |

The data in Table I highlights a few advantages associated with practice of the present invention. Generally, at about 20 MMscfd, the system and process produces about 71 barrels of lean oil per day (BPD) having an RVP of about 19 PSIA, and about 103 BPD of NGL having an RVP of about 103 PSIA.

### Example II

In the second example, where the inlet stream included about 85 mole % of carbon dioxide, tower 82 was configured with 24 ideal trays, the feed point was tray 1, the condenser duty (i.e. propylene refrigeration) was about 7.4 MMBtu/hr., and the reboiler duty was about 5.92 MMBtu/hr. The bottom temperature of tower 82 was 279° F. NGL was drawn as a side draw from tray 21. The system was configured to receive the inlet stream at about 20 MMscfd.

TABLE II

| | | | Condensed | Partially | | | Side | |
|---|---|---|---|---|---|---|---|---|
| | | Partially | Water | Condensed & | Dehydrated, | | Draw | Tower |
| Stream | Inlet | Dehydrated | Stream | Dehydrated | Condensed | Overhead | NGL | Bottoms |
| (Conduit #) | (21) | (35) | (33) | (55) | (71) | (93) | (97) | (95) |
| Rate | 20.02 | 19.93 | 0.09 | 19.9 | 8.68 | 19.15 | — | — |
| (MMscfd) | | | | | | | | |
| Temp (° F.) | 90 | 60 | 60 | −35 | −35 | −31.5 | 80 | 115 |
| Pressure | 214.7 | 204.7 | 204.6 | 185 | 185 | 182 | 173 | 174 |
| (PSIA) | | | | | | | | |
| Liquid (BPD) | — | — | 37.8 | — | 3634 | — | 124 | 353 |
| RVP (PSIA) | — | — | — | — | — | — | 120 | 19 |

Chemical Composition (Mole %)

| | | | | | | | | |
|---|---|---|---|---|---|---|---|---|
| CO2 | 84.6307 | 84.9892 | 8.6004 | 85.1055 | 89.7700 | 87.6112 | 1.8086 | 0.0000 |
| H2S | 0.0501 | 0.0502 | 0.0141 | 0.0503 | 0.0627 | 0.0449 | 0.7671 | 0.0000 |
| Nitrogen | 2.0031 | 2.0125 | 0.0219 | 2.0151 | 0.1091 | 2.0749 | 0.0000 | 0.0000 |
| Methane | 7.0108 | 7.0428 | 0.2648 | 7.0522 | 0.9958 | 7.2612 | 0.0000 | 0.0000 |
| Ethane | 1.7026 | 1.7088 | 0.3644 | 1.7112 | 1.2206 | 1.7620 | 0.0000 | 0.0000 |
| Propane | 1.2019 | 1.2029 | 0.9253 | 1.2050 | 1.7549 | 0.8439 | 42.4119 | 0.6200 |
| i-Butane | 0.5008 | 0.4983 | 0.9624 | 0.4995 | 0.9379 | 0.1748 | 25.4558 | 5.3000 |
| n-Butane | 0.6009 | 0.5955 | 1.6399 | 0.5972 | 1.2042 | 0.1400 | 24.2913 | 12.3900 |
| i-Pentane | 0.3505 | 0.3405 | 2.3397 | 0.3421 | 0.7448 | 0.0339 | 2.3761 | 14.4300 |
| n-Pentane | 0.5509 | 0.5289 | 4.9068 | 0.5321 | 1.1786 | 0.0355 | 2.2465 | 23.9100 |
| n-Hexane | 1.0015 | 0.8760 | 26.2562 | 0.8893 | 2.0180 | 0.0179 | 0.5900 | 43.3600 |
| H2O | 0.3942 | 0.1540 | 53.7000 | 0.0000 | 0.0000 | 0.0000 | 0.0000 | 0.0000 |

The data in Table II highlights a few advantages associated with practice of the present invention. Generally, at about 20 MMscfd, the system and process produces about 353 barrels of lean oil per day (BPD) having an RVP of about 19 PSIA, and about 124 BPD of NGL having an RVP of about 120 PSIA.

### Overall System Sensitivity

Further simulations were conducted using the system design employed in Example II except that the temperature of the inlet stream into tower 82 (i.e. the temperature of the partially-condensed & dehydrated stream carried by conduit 55) was set to desired temperatures, and the required condenser duty to achieve these desired temperatures was calculated by simulation. The impact on percent recoveries of hydrocarbon, overall amount of lean oil and NGL, percent hydrocarbon recovery relative to the tower feed, and reboiler duty was then achieved by simulation. The data output from these simulations is summarized in Table III.

TABLE III

| Temperature | | | | | | | | | | Percent of | |
|---|---|---|---|---|---|---|---|---|---|---|---|
| of Partially | | | | | | | | | | Total Inlet | |
| Condensed & | Condenser | | | | | | | Lean | | Feed to | Reboiler |
| Dehydrated | Duty | % Recoveries | | | | | | Oil | NGL | Tower | Duty |
| (55) (° F.) | (MMBtu/hr) | C3% | iC4 | nC4 | iC5 | nC5 | C6 | (BPD) | (BPD) | (%) | (MMBtu/hr) |
| −35 | 7.3 | 33 | 67 | 78 | 91 | 94 | 98 | 353 | 124 | 43.4 | 5.9 |
| −30 | 3.3 | 28 | 58 | 69 | 87 | 91 | 98 | 345 | 110 | 12.55 | 1.9 |
| −25 | 2.5 | 21 | 49 | 61 | 83 | 88 | 97 | 334 | 91 | 6.05 | 1.1 |
| −20 | 2.3 | 17 | 42 | 54 | 79 | 85 | 96 | 326 | 73 | 4.7 | 0.9 |
| −15 | 2.2 | 14 | 36 | 47 | 74 | 81 | 95 | 318 | 57 | 3.9 | 0.8 |
| −10 | 2.1 | 12 | 31 | 42 | 69 | 77 | 94 | 309 | 44 | 3.4 | 0.7 |
| −5 | 2.1 | 10 | 27 | 36 | 64 | 72 | 92 | 298 | 30 | 3 | 0.64 |
| 0 | 2 | 8 | 23 | 32 | 58 | 67 | 90 | 284 | 22 | 2.65 | 0.6 |

Various modifications and alterations that do not depart from the scope and spirit of this invention will become apparent to those skilled in the art. This invention is not to be duly limited to the illustrative embodiments set forth herein.

1. A process for recovering liquid hydrocarbons from a gaseous stream containing the liquid hydrocarbons, the process comprising:
(a) providing a dehydrated gaseous stream including carbon dioxide, methane, ethane, propane, and C4 and heavier hydrocarbons;
(b) cooling the dehydrated gaseous stream to form a condensate stream including C4 and heavier hydrocarbons;
(c) separating at least a portion of the carbon dioxide, methane and ethane from the condensate stream; and
(d) distilling the condensate stream to thereby form a liquid hydrocarbon stream.

2. The process of claim 1, where the condensate stream distilled in said step of distilling is less than 15 vol % of the dehydrated gaseous stream.

3. The process of claim 1, where the liquid hydrocarbon stream is characterized by an RVP (ASTM D-323) of from about 9 to about 30.

4. The process of claim 1, where said step of cooling the dehydrated gaseous stream cools the stream to a temperature of from about −40 to about 5° F.

5. The process of claim 1, where said step of distilling the condensate stream creates an overhead stream, and where said overhead stream is used in said step of cooling the dehydrated stream.

6. The process of claim 1, where said dehydrated gaseous stream is produced by providing a gaseous production stream from an enhanced oil recovery process, and treating the gaseous stream by cooling to thereby condense at least a portion of the water contained therein and separating the same from the gaseous stream.

7. The process of claim 1, where said step of treating the gaseous stream cools the stream to a temperature of from about 30 to about 110° F.

8. The process of claim 1, where said step of distilling includes capturing a side draw that includes propane.

9. The process of claim 1, further combining the step of combining the liquid hydrocarbon stream with another liquid hydrocarbon stream having a lower RVP (ASTM D-323).

10. The process of claim 1, where said step of cooling the dehydrated stream includes cooling the stream by refrigeration.

11. A method for recovering liquid hydrocarbons from a gaseous stream containing the liquid hydrocarbons, the process comprising:
(a) providing a hydrated gaseous stream including water, carbon dioxide, methane, ethane, propane, and C4 and heavier hydrocarbons;
(b) cooling the hydrated gaseous stream to condense a portion of the water within the hydrated gaseous stream to thereby form a cooled hydrated gaseous stream;
(c) separating the condensed water from the cooled hydrated gaseous stream to provide a partially dehydrated stream;
(d) dehydrating the partially dehydrated stream to provide a dehydrated gaseous stream including carbon dioxide, methane, ethane, propane, and C4 and higher hydrocarbon;
(e) cooling the dehydrated gaseous stream to thereby form a condensate stream including C4 and heavier hydrocarbons;
(f) separating a portion of the carbon dioxide, methane, ethane, and propane from the condensate stream; and
(g) distilling the condensate stream to thereby form a liquid hydrocarbon stream.

12. The method of claim 11, where said step of separating the condensed water from the stream reduces the water content of the stream by greater than 40 vol %.

13. The method of claim 11, where the condensate stream distilled in said step of distilling is less than 15 vol % of the dehydrated gaseous stream.

14. The method of claim 11, where the liquid hydrocarbon stream is characterized by an RVP (ASTM D-323) of from about 9 to about 30.

15. The method of claim 11, where said step of cooling the dehydrated gaseous stream cools the stream to a temperature of from about −40 to about 5° F.

16. The method of claim 11, where said step of distilling the condensate stream creates an overhead stream, and where said overhead stream is used in said step of cooling the dehydrated stream.

17. The method of claim 11, where said step of cooling the gaseous hydrated stream cools the stream to a temperature of from about 30 to about 110° F.

18. The method of claim 11, where said step of distilling includes capturing a side draw that includes propane.

19. The method of claim 11, further combining the step of combining the liquid hydrocarbon stream with another liquid hydrocarbon stream having a lower RVP (ASTM D-323).

20. The method of claim 11, where said step of cooling the dehydrated stream includes cooling the stream by refrigeration.
